(19) United States
(12) Plant Patent Application Publication
(10) Pub. No.: US 2020/0396879 P1
(43) Pub. Date: Dec. 17, 2020

(54) Grape Plant Named 'Gewurztraminer RJ'

(50) ;

(71) Applicant: Ronald Juneau, Pont-Rouge, CA (US)

(72) Inventor: Ronald Juneau, Pont-Rouge, CA (US)

(21) Appl. No.: 16/873,713

(22) Filed: Jun. 10, 2020

(30) Foreign Application Priority Data
Jun. 11, 2019 (CA) 19-9940

(51) Int. Cl. A01H 6/88

(52) U.S. Cl. PLT/205

(57) ABSTRACT

The invention is a new and distinct variety of Grapes plant named ‘Gewurztraminer RJ’ that is characterized by its grape clusters that are medium-large in size and cylindro-conical in shape, its good cold hardiness, its mature fruit early in the season, its vigorous growth habit with high fruit production, its grapes that are pink-purple in color at full maturity, good disease resistance, its grapes that have thick skins and are resistant to fall frost, and its grapes that produce wine that is rich in aroma and with a fruity taste.

### CROSS-REFERENCE TO A RELATED APPLICATION

This application claims priority to a Canadian plant breeders' rights application filed on Jun. 11, 2019, application No. 19-9940, under 35 U.S.C. 119(f), the entire contents of which is incorporated by reference herein.

Botanical classification: Vitis vinifera.

Variety denomination: ‘Gewurztraminer RJ’.

### BACKGROUND OF THE INVENTION

The present invention relates to a new and distinct cultivar of grape plant botanically known as Vitis vinifera ‘Gewurztraminer RJ’, referred to hereafter by its cultivar name, ‘Gewurztraminer RJ’.

The Inventor discovered the new cultivar in autumn of 1996 as a naturally occurring branch mutation of an unnamed and unpatented proprietary plant of Vitis vinifera from the inventor's breeding program, designated as “Gewurztraminer CL 47” that was growing in a field in St-Ubalde, Québec, Canada.

Asexual propagation of the new cultivar was first accomplished by hardwood stem cuttings in 1999 by the Inventor in Port-Rouge, Québec, Canada. Asexual propagation of the new cultivar by hardwood stem cuttings and tissue culture has determined that the characteristics are stable and true to type in successive generations.

### SUMMARY OF THE INVENTION

The following traits have been repeatedly observed and represent the characteristics of the new cultivar. These attributes in combination distinguish ‘Gewurztraminer RJ’ as a new and unique cultivar of Vitis.
1. ‘Gewurztraminer RJ’ exhibits grape clusters that are medium-large in size and cylindro-conical in shape.
2. ‘Gewurztraminer RJ’ exhibits good cold hardiness.
3. ‘Gewurztraminer RJ’ exhbits mature fruit early in the seaon.
4. ‘Gewurztraminer RJ’ exhibits a vigorous growth habit with high fruit production.
5. ‘Gewurztraminer RJ’ exhibits grapes that are pink-purple in color at full maturity.
6. ‘Gewurztraminer RJ’ exhibits good disease resistance.
7. ‘Gewurztraminer RJ’ exhibits grapes that have thick skins and are resistant to fall frost.
8. ‘Gewurztraminer RJ’ exhibits grapes that produce wine that is rich in aroma and with a fruity taste.

The parent plant of ‘Gewurztraminer RJ’, differs from ‘Gewurztraminer RJ’ in having much lesscold hardiness, in being more susceptible to disease, grape clusters that are smaller in size with less weight per cluster, a different leaf shape and grapes that are different in color. ‘Gewurztraminer RJ’ can also be compared to the Viti vinifera cultivars ‘Traminette’ (not patented) and ‘Gewurztraminer CL 643’ (not patented). ‘Traminette’ is similar to ‘Gewurztraminer RJ’ in having grapes that produce wine with a similar structure and mouthfeel. ‘Traminette’ differs from ‘Gewurztraminer RJ’ in having leaves that are different in shape and appearance, grapes that are different in color in clusters that differ in shape, and in being much less cold hardy, in producing grapes later in the season, and in having less disease resisitance. ‘Gewurztraminer CL 643’ is similar to ‘Gewurztraminer RJ’ in having grapes that produce wine that is fragrant, spicy, with good sugar levels, acid levels and PH balance. ‘Gewurztraminer CL 643’ differs from ‘Gewurztraminer RJ’ in having fruit that is different in color, berry size, and cluster size, leaves that are different in shape and leaf buds and young shoots that are different.

### BRIEF DESCRIPTION OF THE DRAWINGS

The accompanying colored photographs illustrate the overall appearance and distinct characteristics of the new grape cultivar. The photographs were taken of plants of the new cultivar as grown outdoors in a field in Saint-Ubalde, Québec, Canada.

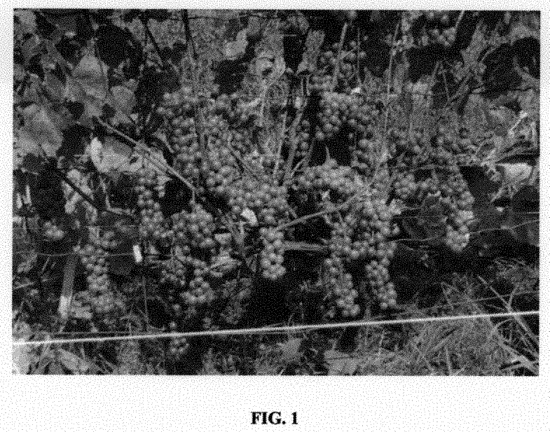

The photograph in FIG. 1 was taken of a plant 5 years in age as grafted onto 3309 rootstock and provides a side view of the plant habit of ‘Gewurztraminer RJ’ with maturing fruit clusters after frost.

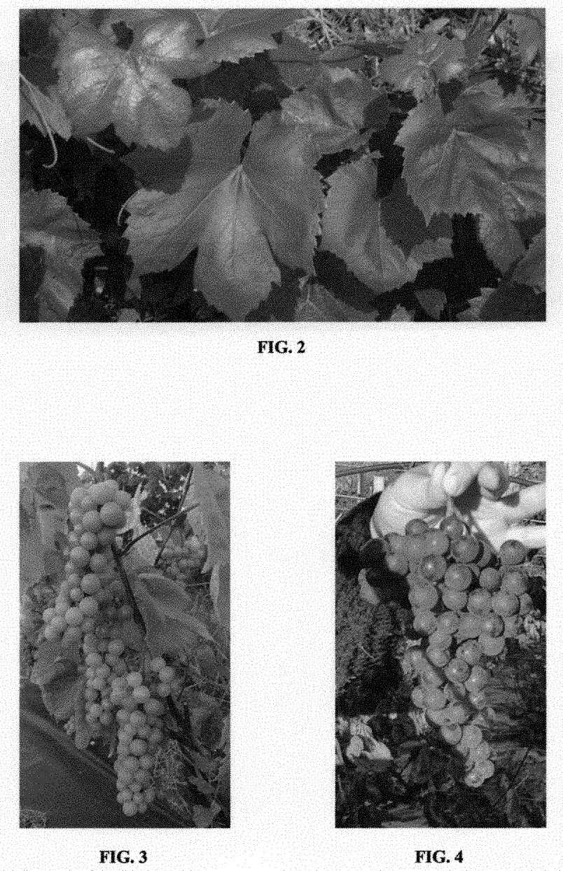

The photograph in FIG. 2 was taken of a plant 9 years in age as grafted onto Vitis riparia rootstock and provides a close-up view of the foliage of ‘Gewurztraminer RJ’.

The photographs in FIG. 3 and FIG. 4 were taken of a plant 3 years in age (not grafted).

The photograph in FIG. 3 provides a close-up view of the immature fruit of ‘Gewurztraminer RJ’.

The photograph in FIG. 4 provides a close-up view of the mature fruit of ‘Gewurztraminer RJ’.

The colors in the photographs are as close as possible with digital photography techniques available, the color values cited in the detailed botanical description accurately describe the colors of the new cultivar of grape.

### DETAILED BOTANICAL DESCRIPTION OF THE PLANT

The following is a detailed description of plants seven years in age as grown outdoors in a trial field in Saint-Ubalde, Québec, Canada. The phenotype of the new cultivar may vary with variations in environmental, climatic, and cultural conditions, as it has not been tested under all possible environmental conditions. The color determination is in accordance with the 2015 Colour Chart of The Royal Horticultural Society, London, England, except where general color terms of ordinary dictionary significance are used.
General description:
   Blooming period.—Typically commences blooms about June 24th in Pontneuf County, Québec, Canada.
   Plant type.—Perennial fruit producing vine.
   Plant habit.—Open but orderly.
   Height and spread.—An average of 1.2 m in height and 220 cm in width.
   Cold hardiness.—At least to U.S.D.A. Zone 4a.
   Diseases and pests.—Resistance to downy mildew (Plasmopara viticola), powdery mildew (Uncinula necator), and black rot (Guignardia bidwellii).
   Propagation.—Hardwood stem cuttings and tissue culture.
   Root description.—Fibrous and fine.
   Root development.—An average of 21 days for root initiation with a young plant produced from a rooted cutting in 45 to 60 days.
   Growth rate.—Vigorous. Additonal detailed boatinical data will be provided by preliminary amendment.

It is claimed:

1: A new and distinct cultivar of Grape plant named ‘Gewurztraminer RJ’ as herein illustrated and described.
